(19) United States
(12) Patent Application Publication
(10) Pub. No.: US 2009/0043270 A1
(43) Pub. Date: Feb. 12, 2009

(54) EFFUSION DRAINAGE KITS AND METHODS FOR PACKAGING THE SAME

(75) Inventors: Jodie L. Noyce, Salt Lake City, UT (US); Richard V. Wretberg, Herriman, UT (US)

Correspondence Address: Rutan & Tucker, LLP., 611 ANTON BLVD, SUITE 1400, COSTA MESA, CA, 92626, US

(73) Assignee: C.R. Bard, Inc., Murray Hill, NJ (US)

(21) Appl. No.: 12/188,955

(22) Filed: Aug. 8, 2008

Related U.S. Application Data
(60) Provisional application No. 60/955,284, filed on Aug. 10, 2007.
(60) Provisional application No. 60/968,465, filed on Aug. 28, 2007.

(51) Int. Cl. A61F 17/00; B65B 11/02

(52) U.S. Cl. 604/327; 206/570; 53/461; 235/375

(57) ABSTRACT

A kit for treating effusion, such as pleural effusion, comprises at least one tray including a plurality of indentations configured to receive one or more components used to insert a chest tube into a patient's pleural cavity, at least one dressing kit including supplies for applying a dressing to the patient's skin surrounding an inserted chest tube, and at least one drainage kit including one or more components for receiving fluid drained from the patient's pleural cavity.

### CROSS-REFERENCE TO RELATED APPLICATIONS

This application claims the benefit of the U.S. Provisional Patent Application Nos. 60/955,284, filed Aug. 10, 2007, entitled “Pleural Effusion Drainage Kits and Methods for Packaging the Same,” and 60/968,465, filed Aug. 28, 2007, entitled “Pleural Effusion Drainage Kits and Methods for Packaging the Same,” both of which are incorporated herein by reference in their entireties.

### BRIEF SUMMARY

The present invention has been developed in response to the above and other needs in the art. Briefly summarized, embodiments of the present invention are directed to a kit for treating effusion occurring within a cavity of a body of a patient. One example of such effusion occurs within the pleural cavity of the patient, where the removal of fluid therefrom is often necessary in order to preserve patient health and comfort. The kit to be described in connection with embodiments of the present invention enables pleural and other effusion treatment to be accomplished in treatment facilities and in-home care situations.

In one embodiment, the kit for treating effusion generally includes an insertion tray, one or more dressing kits, and one or more drainage kits. The insertion tray includes a plurality of indentations configured to receive one or more components used to insert a drainage tube into a patient's pleural or other body cavity where undesired fluid buildup has occurred. The dressing kit(s) include supplies for applying a dressing to the patient's skin surrounding the inserted drainage tube, while the drainage kit(s) include one or more components for receiving fluid drained from the patient's pleural or other body cavity via the drainage tube. The insertion tray, dressing kit(s), and drainage kit(s) are placed in a case and as such are packaged for use by a clinician or other trained person.

In one embodiment, multiple dressing and drainage kits are included in the case together with the insertion tray so as to enable drainage and dressing components to be replaced during the drainage period without requiring additional drainage or dressing supplies to be acquired. In addition, one or more vouchers are included in the case to enable a patient being cared for at home, for example, to conveniently order additional dressing, drainage, or other supplies relating to the effusion treatment kit without requiring a return trip to the treatment facility. Methods related to packaging the effusion treatment kit are also disclosed herein.

These and other features of the present invention will become more fully apparent from the following description and appended claims, or may be learned by the practice of the invention as set forth hereinafter.

### BRIEF DESCRIPTION OF THE DRAWINGS

Advantages of the instant disclosure will become apparent upon review of the following detailed description and drawings, which illustrate representations (not necessarily drawn to scale) of various aspects of the instant disclosure.

FIG. 1 shows a schematic diagram of a kit for treating effusion, such as pleural effusion, according to one embodiment.

FIG. 2 shows a schematic diagram of components that the tray of FIG. 1 may include.

FIG. 3 shows a schematic diagram of components that the dressing kit of FIG. 1 may include.

FIG. 4 shows a schematic diagram of components that the drainage kit of FIG. 1 may include.

FIG. 5 shows a schematic diagram of a case containing the tray, the dressing kit, and the drainage kit of FIG. 1, according to one embodiment.

FIG. 6 shows a schematic diagram of a case containing the tray, the dressing kit, and the drainage kit of FIG. 1, according to another embodiment.

FIG. 7 shows a schematic diagram of an in-home drainage kit according to one embodiment.

FIG. 8 shows a schematic diagram of a case containing the dressing kit and the drainage kit of FIG. 1, according to one embodiment.

FIG. 9 shows a flow chart for a method of packaging an effusion insertion tray, according to one embodiment.

FIG. 10 shows a flow chart for a method of packaging an effusion insertion tray, according to another embodiment.

### DETAILED DESCRIPTION

With reference to the above-listed drawings, this section describes particular embodiments and their detailed construction and operation. The embodiments described herein are set forth by way of illustration only. Those skilled in the art will recognize in light of the teachings herein that variations can be made to the embodiments described herein and that other embodiments are possible. No attempt is made to exhaustively catalog all possible embodiments and all possible variations of the described embodiments.

For the sake of clarity and conciseness, certain aspects of components or steps of certain embodiments are presented without undue detail where such detail would be apparent to those skilled in the art in light of the teachings herein and/or where such detail would obfuscate an understanding of more pertinent aspects of the embodiments.

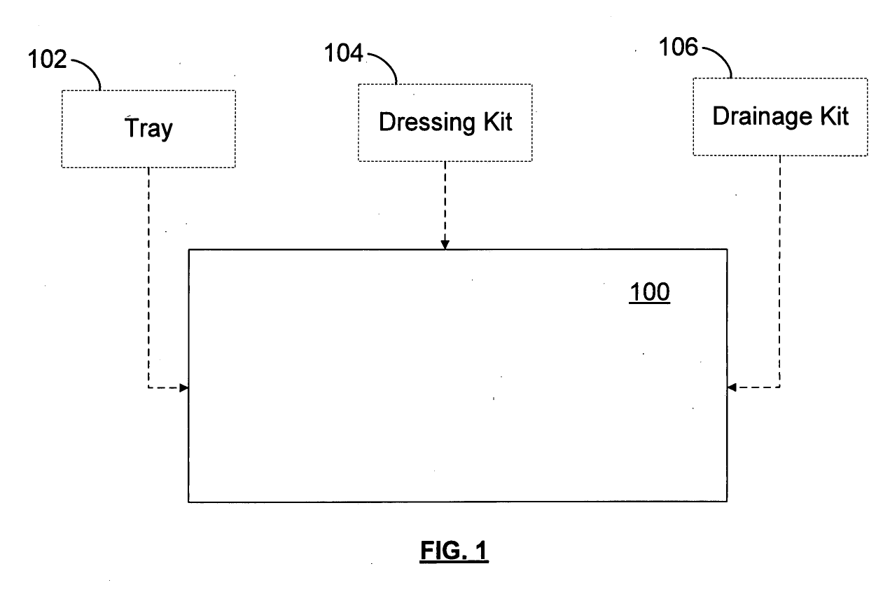

FIG. 1 illustrates a kit 100 that may be used for treating effusion in a patient, including for example, pleural effusion. Pleural effusion is a buildup of fluid between an outer lining of a patient's lungs (the patient's visceral lining) and an inner lining of the patient's chest cavity. Pleural fluid, in small amounts, helps lubricate the surface of the pleura (e.g., during respiration). However, an abnormal collection of pleural fluid results in pleural effusion, the causes of which are many. For example, the patient may have congestive heart failure generating an abnormal lung pressure or the patient may have a lung disease, such as lung cancer, pneumonia, or tuberculosis, which causes inflammation of the pleura. The kit 100 may be used by a medical professional and/or the patient to drain excess pleural fluid from the patient's pleural cavity.

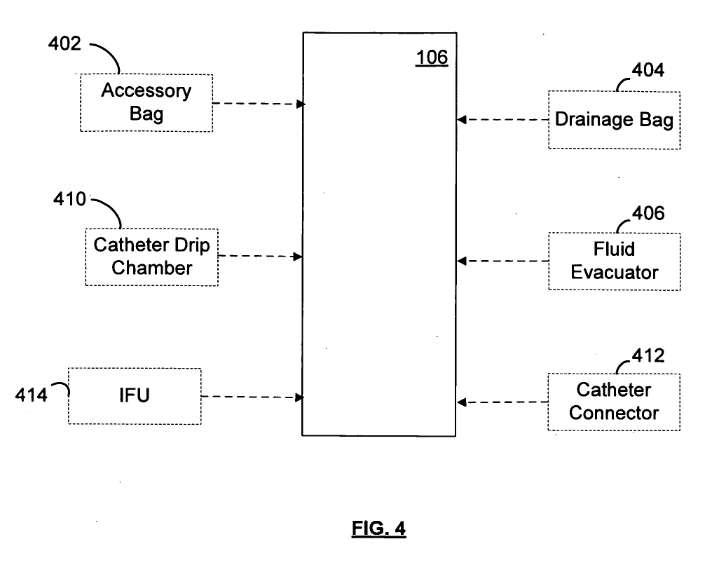

The kit 100 may include a sealed tray 102 that includes a plurality of indentations for receiving components used to insert a chest tube into the patient's pleural cavity. In addition, the kit 100 may include one or more dressing kits 104 for applying a dressing to the patient's skin surrounding the inserted chest tube. One or more drainage kits 106 may be provided with the kit 100 to collect fluid drained from the patient's pleural cavity. The drainage kit 106 may allow the patient to replace a full drainage bag 404 (FIG. 4) with another draining bag 404 to collect more fluid without visiting a medical professional (or having the medical professional visit the patient's home). This may not only help the patient stay healthier (by avoiding trips to the hospital filled with ill patients), but may also save the patient time and money.

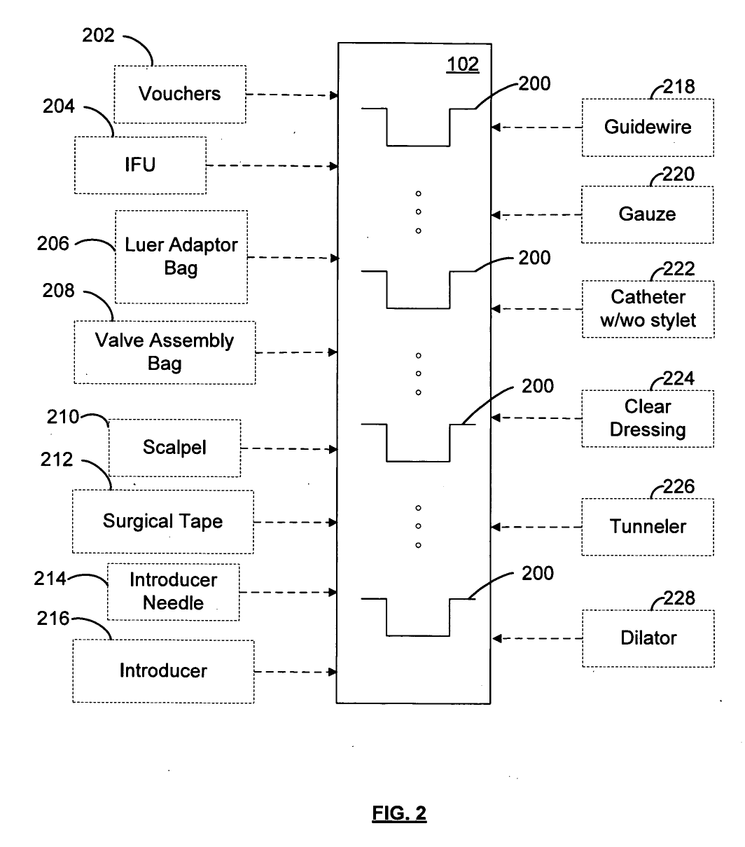

FIG. 2 illustrates various components that the tray 102 may contain. The tray 102 may be molded from plastic, such as polyvinyl chloride, polypropylene, or silicone and may include indentations 200 sized to accommodate one or more of the components used to insert the chest tube into the patient's pleural space. For example, one or more vouchers 202 may be included in the tray so that the patient can order additional drainage kits 106. Instructions for use (IFU) 204 may also be included to describe using all or part of kit 100 and/or the components in the tray 102. The kit 100 may also include one or more luer adaptor bags 206 and one or more valve assembly bags 208. The luer adaptor bag 206 may include a luer adapter and luer adapter instructions that may be used for attaching a catheter 222, with or without a stylet, to the drainage bag 404. The valve assembly bag 208 may include a valve assembly, a side clamp, a valve clamp, a valve cap, and valve assembly instructions for use that may be used to control the flow of fluid from the pleural cavity.

The tray 102 may further include a scalpel 210 to make an incision in the patient's skin and a dilator 228 to dilate the subcutaneous tissues. In addition, a tunneler 226 (with or without protective covers), introducer needle 214, introducer 216, and guidewire 218 may be included to facilitate inserting the catheter 222 into the pleural cavity. Further, surgical tape 212, gauze 220, and clear or semitransparent dressing 224 may be provided to dress the skin surrounding the inserted catheter 222.

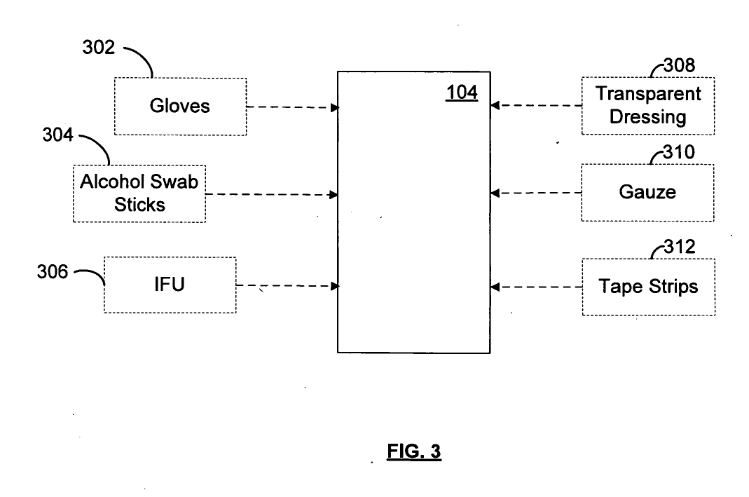

With reference to FIG. 3, the dressing kit 104 may include a number of supplies to apply a dressing to the patient's skin surrounding the inserted chest tube. The dressing kit 104 may be used by the medical professional immediately following the insertion procedure, or the dressing kit 104 may be used by the patient to periodically change the dressing surrounding the inserted chest tube. According to one embodiment, the dressing kit 104 includes sterilized gloves 302, alcohol swabs 304, information for use 306 describing the dressing process, transparent dressing 308, gauze 310, and tape strip 312. Additional or fewer supplies may be included with the dressing kit 104.

Referring now to FIG. 4, the drainage kit 106 may include an accessory bag 402 that may include a valve cap, slide clamp, tape strips, and alcohol prep pads. The slide clamp may be used to stop or slow the pleural fluid flowing from the catheter 222 in the event that the catheter 222 or valve may become damaged. The drainage bag 404 may be any bag suitable to retain the fluid flowing from the pleural cavity and may be sized to hold various amounts of fluid. A fluid pump chamber or evacuator 406 may be included between the drainage bag 404 and the catheter 222 to help pump fluid out of the pleural cavity. In addition, the drainage kit 106 may include a catheter drip chamber 410, a connector 412 configured to receive the catheter 222, and instructions for use 414.

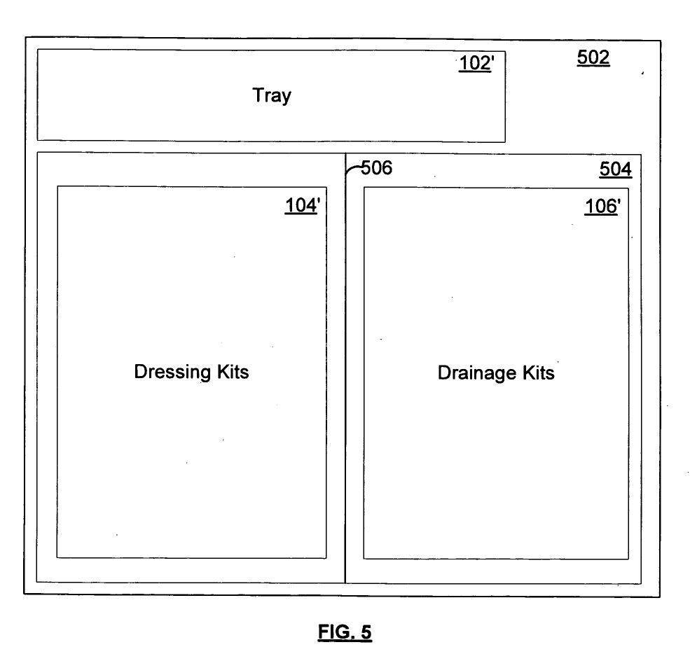

With reference to FIG. 5, the packing of the tray 102′, dressing kits 104′, and drainage kits 106′ is shown according to one embodiment (in FIG. 5 reference numerals with the prime symbol, e.g., 102′, indicate elements similar to those of the same name in another embodiment, e.g., the tray 102 illustrated in FIG. 1). A dispenser box 504 may include a partition 506 to help keep the dressing kits 104′ and drainage kits 106′ separate. According to one embodiment, one or more dressing kits 104′, such as twenty dressing kits 104′, and one or more drainage kits 106′, such as twenty drainage kits 106′, are placed in the dispenser box 504 on respective sides of the partition 506. The dispenser box 504 may then be placed in a case 502 along with one or more trays 102′. The dispenser box 504 and/or the case 502 may be made from any suitable material, such as cardboard.

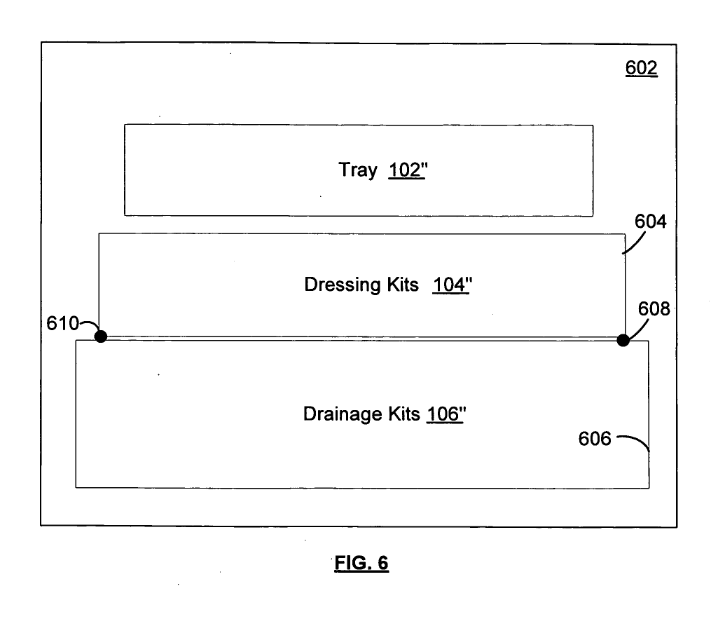

With reference to FIG. 6, a configuration of tray 102″, dressing kits 104″, and drainage kits 106″ is shown according to another embodiment (in FIG. 6 reference numerals with the prime symbol, e.g., 102″, indicate elements similar to those of the same name in another embodiment, e.g., the tray 102 illustrated in FIG. 1). According to this embodiment, one or more dressing kits 104″, such as five dressing kits 104″, are placed in a dressing kit carton 604 and one or more drainage kits 106″, such as five drainage kits 106″, are placed in a drainage kit carton 606. The dressing kit carton 604 may be secured to the drainage kit carton 606, such as, for example, by placing glue dots 608 and 610 on each corner of the dressing kit carton 604 and pressing the dressing kit carton 604 onto the drainage kit carton 606. The dressing kit carton 604, the drainage kit carton 606, and one or more trays 102″ may be placed in a case 602. While FIGS. 5 and 6 discuss examples of packing the trays, dressing kits, and drainage kits, other configurations are possible.

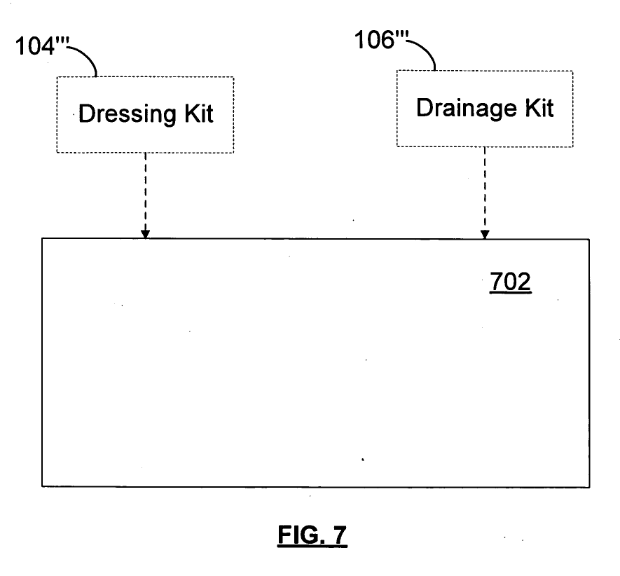

An in-home drainage kit 702 for receiving fluid drained from a patient's pleural cavity is illustrated in FIG. 7, according to one embodiment. The in-home drainage kit 702 may include one or more dressing kits 104′″ and/or one or more drainage kits 106′″ (in FIG. 6 reference numerals with the prime symbol, e.g., 106′″, indicate elements similar to those of the same name in another embodiment, e.g., the drainage kit 106 illustrated in FIG. 1). As previously described with reference to FIG. 4, the drainage kit 106′″ may include one or more drainage bags 404 which may be configured to attach to a distal end portion of the chest tube so that an interior chamber of the drainage bag 404 is in fluid communication with the patient's pleural space. Other supplies and components described with reference to FIGS. 3 and 4 may also be provided with the in-home drainage kit 702.

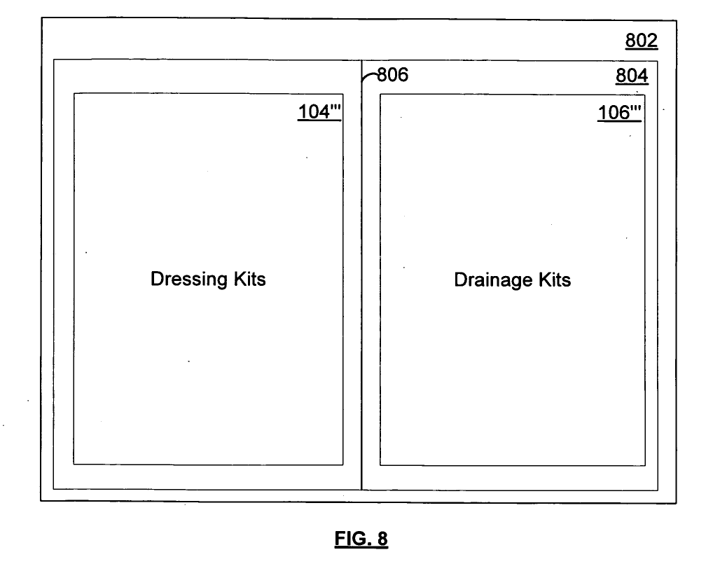

FIG. 8 illustrates the packaging of the dressing kits 104′″ and/or the drainage kits 106′″ according to one embodiment. Similar to the embodiment described in FIG. 5, a dispenser box 804 may include a partition 806 to help keep the dressing kits 104′″ and the drainage kits 106′″ separate. According to one embodiment, one or more dressing kits 104′″, such as twenty dressing kits 104′″, and one or more drainage kits 106′″, such as twenty drainage kits 106′″, are placed in the dispenser box 804 on respective sides of the partition 806. The dispenser box 804 may then be placed in a case 802 which may then be shipped directly to the patient for use. Accordingly, the patient may replace the drainage bag when full and dress their skin surrounding the inserted chest tube without visiting a medical professional. While FIG. 8 discusses an example of packing the trays, dressing kits, and drainage kits, other configurations are possible, such as that described with reference to FIG. 6.

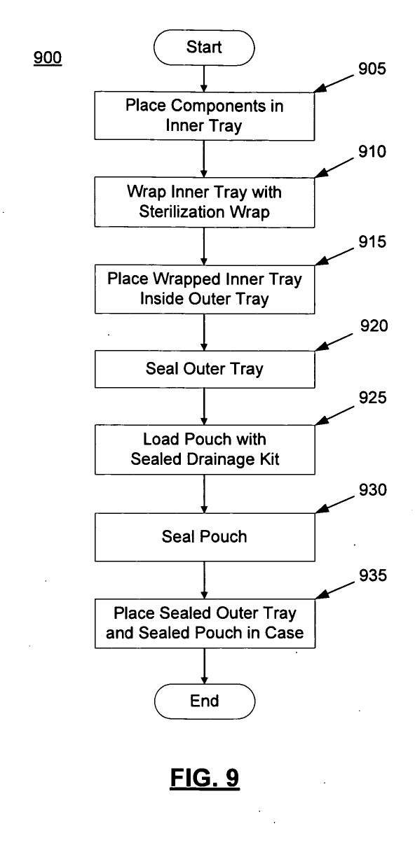

Referring now to FIG. 9, a method 900 of packaging a pleural effusion insertion tray will be described according to one embodiment. At step 905, components used to insert a chest tube into a patient's pleural cavity, such as those described with reference to FIG. 2, are placed into corresponding indentations in an inner tray. By way of example, a luer adapter and luer adapter instructions for use may be placed in a luer adaptor bag which may be placed in the inner tray before wrapping the inner tray. By way of another example, a valve assembly, a side clamp, a valve clamp, a valve cap, and valve assembly instructions for use may be placed in a valve assembly bag which itself may be placed in the inner tray before wrapping the inner tray.

The packager may look for and remove visible particulates from the inner tray. In one embodiment, this is done without the use of alcohol soaked wipes or adhesive. At step 910, the inner tray is wrapped with a sterilization wrap resistant to bacterial penetration. For example, a blue central supply room (CSR) wrap may be used. CSR wrap is generally non-woven, non-absorbent, and resistant to bacterial penetration due to its non-woven construction. According to one embodiment, the tray may be sterilized (e.g., using steam, ethylene oxide sterilization, or gamma radiation) after being wrapped. At step 915, the wrapped inner tray is placed inside an outer tray and a lid is hermetically sealed to the outer tray at step 920. The outer tray lid in one embodiment can be formed from high-density polyethylene, such as that manufactured under the mark TYVEK®, though other materials for this and the protective materials for the drainage and dressing kits can also be employed.

According to one embodiment, the packager ensures that the wrap of the inner tray does not encroach over the portion of the outer tray to which the outer tray lid is sealed and ensures that that the outer tray is in good condition. If the outer tray appears to be defective, it is discarded and replaced. Defects on the outer tray may include warped or curled flanges, pinholes, kinks, bends, contamination, stains or any other deformation in the body or in the flange of the outer tray. A similar inspection can also be performed on the inner tray.

Certain kits may include vouchers for use by the patient for ordering additional drainage kits. The packager can scan the vouchers according to the following method. The packager first turns on a scanner and enters an employee number into an ID field (such as by scanning their badge). Next, a lot number associated with a work order may be entered into an archive number field (such as by scanning the lot number on the work order). Each voucher may then be scanned using the scanner so that the scanned voucher data can be associated with the scanned work order data in a centralized database. If needed, the packager logs onto a network. If a voucher was inadvertently added, it may be removed by pressing a remover button and rescanning the voucher. After the vouchers are scanned, the data is stored in the database.

At step 925, a pouch may be loaded with a sealed tray drainage kit including components used to receive fluid drained from the patient's pleural cavity (e.g., those components described with reference to FIG. 4). If the drainage kit includes an accessory bag, a valve cap, slide clamp, tape strips, and alcohol prep pads may be placed in the accessory bag which can itself be placed in the pouch. At step 930, the pouch may be hermetically sealed and the packager may inspect the pouch to ensure it has a continuous seal. At step 935, the hermetically sealed outer tray and the hermetically sealed pouch may be placed in a case.

According to some embodiments, instructions for use describing a method for using all or part of the kit may be placed in the case. In addition, sterile gloves, alcohol swabs, sterile gauze dressings, tape strips, and instructions for use may be placed in a dressing kit which can be placed in the pouch together with the drainage kit.

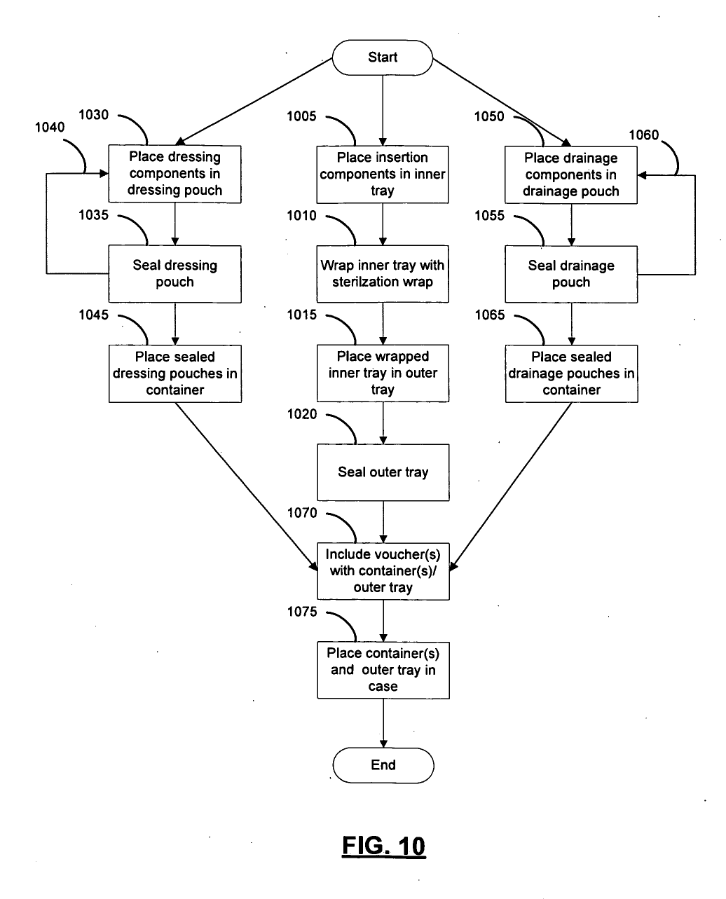

FIG. 10 depicts a method 1000 for packaging a pleural effusion insertion tray and associated components. Several of the steps depicted in FIG. 10 are similar to those discussed in connection with FIG. 9; as such, only selected aspects of the method 1000 are discussed in detail below.

At step 1005, components used to insert a chest tube into a patient's pleural cavity, such as those described with reference to FIG. 2, are placed into corresponding indentations in an inner tray. After inspection thereof, the inner tray is wrapped in step 1010 with a sterilization wrap resistant to bacterial penetration, such as CSR wrap or other suitable protective barrier. Sterilization of the inner tray may then take place, noting that further sterilization of the components of the pleural effusion drainage kit may occur later. Note that, in one embodiment, the inner tray has no indentations.

At step 1015, the wrapped inner tray is placed inside an outer tray and a lid is hermetically sealed to the outer tray at step 920. Again, the outer tray lid in one embodiment is formed from high-density polyethylene, such as that manufactured under the mark TYVEK®.

Steps 1030-1045 and steps 1050-1065 can be performed before, after, or simultaneous to steps 1005-1020 described above. In particular, steps 1030-1045 describe packaging of supplies for applying a dressing to a patient's skin surrounding an inserted drainage chest tube. In step 1030, components used to apply a dressing to a patient's skin surrounding the insertion site of the drainage chest tube, such as those components described with reference to FIG. 3, are placed into a dressing pouch or other suitable enclosure. At step 1035, the dressing pouch is hermetically sealed using high-density polyethylene, such as that manufactured under the mark TYVEK®, or other suitable protective barrier. At step 1040, the pouch placement and sealing of steps 1030 and 1035 are repeated as necessary until a desired number of sealed dressing pouches, each containing the various dressing components, are produced.

At step 1045, the sealed dressing pouches are placed in a container. In one embodiment, the container is a dispenser box, such as the dispenser box 504 shown in FIG. 5, for example. In another embodiment, the container is a carton, such as that shown at 604 in FIG. 6.

Steps 1050-1065 describe packaging of supplies for receiving fluid drained from a patient's pleural cavity or other body cavity. In step 1050, components used for receiving fluid drained from the patient's pleural cavity, such as those components described with reference to FIG. 4, are placed into a drainage pouch or other suitable enclosure. At step 1055, the drainage pouch is hermetically sealed using high-density polyethylene, such as that manufactured under the mark TYVEK®, or other suitable protective barrier. At step 1060, the pouch placement and sealing of steps 1050 and 1055 are repeated as necessary until a desired number of sealed drainage pouches, each containing the various drainage components, are produced.

At step 1065, the sealed drainage pouches are placed in a container. In one embodiment, the container is a dispenser box, such as the dispenser box 504 shown in FIG. 5, for example. In this case, the drainage pouches are placed in a dispenser box that also contains a plurality of dressing pouches, separated by a partition as in FIG. 5, for example. In another embodiment, the container is a carton, such as that shown at 606 in FIG. 6.

At step 1070, one or more vouchers to be redeemed by the patient to order additional drainage kits are included. In one embodiment, the voucher(s) are enclosed in a “kangaroo pouch,” which is affixed to the sealed outer tray including the components used to insert a chest tube into a patient's pleural cavity. In another embodiment, the kangaroo pouch containing the vouchers is affixed to the dispenser box containing the dressing and drainage pouches, or to one of the cartons containing the dressing pouches or drainage pouches. In yet other embodiments, no kangaroo pouch is used, and the vouchers are simply included in a case together with the trays and pouches. Or, the vouchers are affixed directly to the interior or exterior of the case. These and other possible voucher placement locations are therefore contemplated as residing within the claims of the present invention. Preparation, scanning, and recording of the vouchers can proceed as outlined in the above discussion relating to FIG. 9.

At step 1075, the container(s) containing the dressing and drainage pouches and the sealed outer tray including the components used to insert a chest tube into a patient's pleural cavity are placed in a case made from cardboard or other suitable component. The case can then be subjected to further processing as needed and/or prepared for shipment to its intended destination. It should be appreciated that, while the above discussion has focused on use of the present system for drainage of pleural effusion, other patient drainage needs can be addressed by the present system including, for instance, peritoneal drainage.

The terms and descriptions used herein are set forth by way of illustration only and are not meant as limitations. Those skilled in the art will recognize that many variations can be made to the details of the above-described embodiments without departing from the underlying principles of the invention. The scope of the invention should therefore be determined only by the following claims (and their equivalents) in which all terms are to be understood in their broadest reasonable sense unless otherwise indicated.

What is claimed is:

1. A kit for treating pleural effusion, comprising:
at least one tray including a plurality of indentations configured to receive one or more components used to insert a chest tube into a patient's pleural cavity;
at least one dressing kit including supplies for applying a dressing to the patient's skin surrounding an inserted chest tube; and
at least one drainage kit including one or more components for receiving fluid drained from the patient's pleural cavity.

2. The kit of claim 1 further comprising:
one or more vouchers for use by the patient to order additional drainage kits.

3. The kit of claim 1 further comprising:
instructions for use describing a method for using all or part of the kit.

4. The kit of claim 1 wherein the one or more components used to insert the chest tube into the patient's pleural cavity include a storage bag containing a luer adapter and luer adapter instructions for use.

5. The kit of claim 1 wherein the one or more components used to insert the chest tube into the patient's pleural cavity include a storage bag containing a valve assembly, a side clamp, a valve clamp, a valve cap, and valve assembly instructions for use.

6. The kit of claim 1 wherein the one or more components used to insert the chest tube into the patient's pleural cavity include a catheter, a dilator, an introducer, an introducer needle, a tunneler with protective covers, a guidewire, a scalpel, gauze, clear dressing, and/or surgical tape.

7. The kit of claim 1 wherein the one or more components for receiving fluid drained from the patient's pleural cavity include a storage bag containing a valve cap, slide clamp, tape strips, and/or alcohol prep pads.

8. The kit of claim 1 wherein the one or more components for receiving fluid drained from the patient's pleural cavity include a sealed pouch containing a drainage bag, a fluid evacuator, a catheter drip chamber, a connector configured to receive a catheter, and/or instructions for use.

9. The kit of claim 1 wherein the supplies for applying the dressing to the patient's skin surrounding the inserted chest tube include sterile gloves, alcohol swabs, sterile gauze dressings, tape strips, and/or instructions for use.

10. The kit of claim 1 further comprising:
a dispenser box including a partition dividing the dispenser box into first and second portions, the first portion of the dispenser box sized to accommodate twenty dressing kits, and the second portion of the dispenser box sized to accommodate twenty drainage kits; and
a case sized to accommodate the dispenser box and the at least one tray.

11. The kit of claim 1 further comprising:
a dressing kit carton sized to accommodate five dressing kits;
a drainage kit carton sized to accommodate five drainage kits, the dressing kit carton secured to the drainage kit carton; and
a case sized to accommodate the drainage kit carton, the dressing kit carton, and the at least one tray.

12. An in-home drainage kit for receiving fluid drained from a patient's pleural cavity, the in-home drainage kit comprising:
at least one drainage bag configured to attach to a distal end portion of a chest tube so that an interior chamber of the drainage bag is in fluid communication with the patient's pleural cavity; and
one or more vouchers for use by the patient to order additional in-home drainage kits.

13. The kit of claim 12 further comprising:
a storage bag containing one or more of the following: a valve cap, slide clamp, tape strips, and alcohol prep pads.

14. The kit of claim 12 further comprising:
a sealed pouch containing the at least one drainage bag, a fluid evacuator, a catheter drip chamber, a connector configured to receive a catheter, and/or instructions for use.

15. The kit of claim 12 further comprising:
at least one dressing kit including supplies for applying a dressing to the patient's skin surrounding an inserted chest tube.

16. The kit of claim 12 further comprising:
a dispenser box including a partition dividing the dispenser box into first and second portions, the first portion of the dispenser box sized to accommodate ten dressing kits, and the second portion of the dispenser box sized to accommodate ten drainage kits.

17. A method of packaging an effusion draining kit, comprising:
placing components used to insert a drainage tube into a patient's body cavity into corresponding indentations in an inner tray;
wrapping the inner tray with a sterilization wrap resistant to bacterial penetration;
placing the wrapped inner tray inside an outer tray;
hermetically sealing a lid to the outer tray;
loading a pouch with a drainage kit including components used to receive fluid drained from the patient's body cavity;
hermetically sealing the pouch; and
placing the hermetically sealed outer tray and the hermetically sealed pouch in a case.

18. The method of claim 17 further comprising:
scanning a first barcode on a voucher, the voucher for use by the patient to order an additional drainage kit;
scanning a second barcode associated with a work order;
associating the scanned voucher data with the scanned work order data in a centralized database; and
placing the voucher in the case.

19. The method of claim 17 further comprising:
placing instructions for use describing a method for using all or part of the kit in the case.

20. The method of claim 17 further comprising:
placing a luer adapter and luer adapter instructions for use in a storage bag; and
placing the storage bag in the inner tray before wrapping the inner tray.

21. The method of claim 17 further comprising:
removing particulates from the inner tray before wrapping the inner tray;
inspecting the outer tray for defects; and
inspecting the pouch for a continuous seal.

22. The method of claim 17 further comprising:
dividing a dispenser box into first and second portions;
placing twenty dressing kits in the first portion of the dispenser box;
placing twenty drainage kits in the second portion of the dispenser box; and
placing the dispenser box along with the hermetically sealed outer tray in the case.

23. The method of claim 17 further comprising:
placing five dressing kits in a dressing kit carton;
placing five drainage kits in a drainage kit carton;
securing the dressing kit carton to the drainage kit carton; and
placing the drainage kit carton and the dressing kit carton along with the hermetically sealed outer tray in the case.

24. A method of packaging an effusion draining kit, the method comprising:
placing components used to insert a drainage tube into a body cavity of a patient in an inner tray;
wrapping the inner tray with a sterilization wrap resistant to bacterial penetration;
placing the wrapped inner tray inside an outer tray;
hermetically sealing the outer tray; and
placing the outer tray and at least one voucher for ordering additional supplies relating to the effusion draining kit in a case.

25. The method of claim 24 further comprising:
placing dressing components used to apply a dressing to a patient's skin surrounding an insertion site of the drainage tube in a dressing pouch;
hermetically sealing the dressing pouch;
repeating the placing of dressing components and hermetically sealing steps to produce a plurality of sealed dressing pouches; and
placing the plurality of sealed dressing pouches in the case.

26. The method of claim 25 further comprising:
placing drainage components used for receiving fluid drained from the body cavity of the patient in a drainage pouch;
hermetically sealing the drainage pouch;
repeating the placing of drainage components and hermetically sealing steps to produce a plurality of sealed drainage pouches; and
placing the plurality of sealed drainage pouches in the case.

27. The method of claim 26 wherein placing the at least one voucher further comprises enclosing the at least one voucher in a kangaroo pouch.

28. The method of claim 27 wherein the pluralities of sealed dressing pouches and drainage pouches are placed in a dispenser box, the dispenser box including a partition separating the dressing pouches from the drainage pouches, the dispenser box being placed in the case.

30. The method of claim 28 wherein placing the at least one voucher further comprises attaching the kangaroo pouch to one of the outer tray and the dispenser box.

31. The method of claim 27 wherein the plurality of sealed dressing pouches are placed in a dressing carton, the dressing carton being placed in the case, and wherein the plurality of sealed drainage pouches are placed in a drainage carton, the drainage carton being placed in the case.

32. The method of claim 31 wherein placing the at least one voucher further comprises attaching the kangaroo pouch to one of the outer tray, the dressing carton, and the drainage carton.
